(19) United States
(12) Patent Application Publication
(10) Pub. No.: US 2010/0204587 A1
(43) Pub. Date: Aug. 12, 2010

(54) Wavelength Division Sensing Method and Apparatus for Doppler Radar Vital Sign Monitoring and Mechanical Vibration Monitoring

(75) Inventors: Jenshan Lin, Gainesville, FL (US); Changzhi Li, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: University of Florida Research Foundation, Inc., Gainesville, FL (US)

(21) Appl. No.: 12/668,473

(22) PCT Filed: Jul. 10, 2008
(86) PCT No.: PCT/US08/69692

§ 371 Date: Jan. 11, 2010

Related U.S. Application Data
(60) Provisional application No. 60/948,749, filed on Jul. 10, 2007.

(51) Int. Cl. A61B 5/0205; G06F 19/00

(52) U.S. Cl. 600/484; 702/56

(57) ABSTRACT

Embodiments of the present invention provide a method and a radar system incorporating multiple carrier wavelengths. A multi-carrier radar method and system according to the present invention can be used to realize sensing of complex pattern vibrations using a wavelength division sensing technique.

### CROSS-REFERENCE TO RELATED APPLICATION

The present application claims the benefit of U.S. Application Ser. No. 60/948,749, filed Jul. 10, 2007, which is hereby incorporated by reference herein in its entirety, including any figures, tables, or drawings.

### BACKGROUND OF INVENTION

Microwave Doppler radar can be used for non-contact, through-clothing measurement of chest wall motion, from which heart and respiration signatures and rates can be derived in real-time. The microwave spectrum encompasses electromagnetic (EM) energy in frequencies from 1 GHz-1000 GHz. The microwave spectrum is partitioned into bands. The IEEE US radio band designations include L band for 1-2 GHz, S band for 2-4 GHz, C band for 4-8 GHz, X band for 8-12 GHz, Ku band for 12-18 GHz, K band for 18-26 GHz, Ka band for 26-40 GHz, and V band for 40-75 GHz. Typical monitoring systems that use microwave Doppler radar send an EM wave of one wavelength.

### BRIEF SUMMARY

Embodiments of the present invention provide a method and a radar system incorporating multiple carrier wavelengths. A multi-carrier radar method and system according to the present invention can be used to realize sensing of complex pattern vibrations using a wavelength division sensing technique.

### BRIEF DESCRIPTION OF DRAWINGS

FIG. 1 shows a schematic overview of a system using a wavelength division sensing technique according to an embodiment of the present invention, where signals having different wavelengths are generated, transmitted, and detected.

### DETAILED DISCLOSURE

An embodiment of the present invention provides a non-contact Doppler radar method and sensing system for the monitoring of mechanical vibration. In a specific embodiment of the present invention, a non-contact Doppler radar sensing method and system for the monitoring of vital signs is provided. In embodiments of the present invention, electromagnetic (EM) waves of at least two different wavelengths can be transmitted simultaneously in a non-contact Doppler radar sensing system for the monitoring of vital sign and/or mechanical vibration. The EM waves can be RF signals. In a specific embodiment, continuous wave (CW) RF signals are utilized. Vital signs of a human or animal can be monitored. In a specific embodiment, two or more vital signs and/or mechanical vibrations can be monitored simultaneously. In order to monitor two or more vibrations simultaneously, the two or more RF transmitters can transmit simultaneously and the two or more RF receivers can receive simultaneously. In another embodiment, two or more vital signs can be monitored sequentially. In order to monitor sequentially, two or more vibrations sequentially, the two or more RF transmitters can transmit sequentially and the two or more RF receivers can receive sequentially.

Certain frequencies can be better for detecting specific vital signs than other frequencies. For example, short wavelengths can be preferred for use to detect cardiopulmonary activities, and long wavelengths can be preferred for use to detect large muscular activities such as those associated with respiration. Preferably, wavelengths are used such that the amplitude of the vibration being monitored is in the range λ/20 to λ/5, and preferably around λ/10, where 2 is the wavelength of the RF signal. In various embodiments, the RF signal is transmitted for at least one period of the vibration, two periods of the vibration, three periods of the vibration, and, for more accurate measurements, up to 20 periods or more of the vibration.

According to an embodiment of the present invention, detected signals using different wavelengths can be analyzed together to enhance a system's detection capability and accuracy. In one embodiment, a combination of short wavelength and long wavelength radar can be used in a vital sign monitoring system. The received signals can be analyzed together using a wavelength division technique.

When used to monitor mechanical vibration, different wavelengths induce different frequency components of a detected signal, rendering it possible to extract the harmonic caused by the movement itself. Techniques for extracting vibrational information based on harmonics are taught in International Application No. PCT/US2008/065550, filed Jun. 2, 2008, which is hereby incorporated by reference in its entirety. Therefore, embodiments of the present invention can extend the capacity of mechanical vibration monitoring from single frequency sinusoidal vibration to complex pattern vibration. The extraction of the signal information from the complex pattern vibration can be referred to as a wavelength division sensing technique. A multi-carrier radar system can be used to realize this wavelength division sensing technique.

Embodiments of the subject invention can be used, for example, in healthcare monitoring systems, biomedical sensors, lie-detection systems, military personal radar carried by soldiers for behind-the-wall sensing, and security systems, by providing information regarding motion of objects and/or heartbeat and/or breathing, or other vibration, of a person. Further embodiments can be used, for example, in industrial applications in factory production lines, mechanical vibration monitoring systems, the aeronautics and aerospace industry, periodic movement monitoring systems, and actuator calibration systems by providing information regarding motion and/or vibrations of objects. Further embodiments relate to entertainment/gaming applications, such as video games. Specific embodiments can detect breathing and/or heart rate, and/or other motion information regarding a person playing the video game and feed that information into the video game. All of the above systems can be non-contact systems. In an embodiment, the system can be made portable.

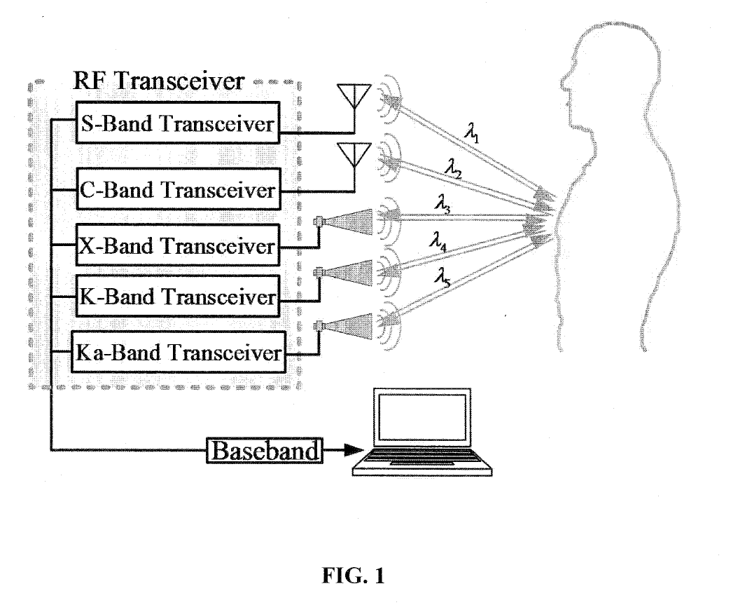

An embodiment of the present invention can incorporate transceivers of different wavelengths. In alternative embodiments, separate transmitters and receivers can be utilized. Referring to FIG. 1, signals with different wavelengths λ₁, λ₂, λ₃, λ₄, and λ₅ can be generated, and can be transmitted and received through different antennas. The antennas can include an S band, C band, X band, K band, and Ka band transceiver.

This technique can enhance a detection system's capability and accuracy. It can also eliminate potential interference between different signals to be detected simultaneously (e.g., heartbeat signal and respiration signal). Further, it can also extend the application of mechanical vibration monitoring systems from the detection of simple sinusoidal movement to the detection of complex movement pattern. Such complex movement patterns can include, but are not limited to, a triangular or square pattern. In a specific embodiment, an RF transmitter can both transmit RF signals, sequentially, and can, optionally, scan the RF wavelength with time. The scanning can be accomplished until a vibration is detected and then the RF transmitter can transmit at the appropriate wavelength for the detected vibration. Another option is the ability to tune the wavelength based on the detected vibration.

Various techniques are known in the art for extracting the vibrational information from the detected reflected RF signal(s). Such techniques include, but are not limited to, the techniques taught in Kun-Mu Chen et al. “Microwave Life-Detection Systems for Searching Human Subjects Under Earthquake Rubble or Behind Barrier,” IEEE transactions on Biomedical Engineering, Vol. 27, No. 1, January 2000, International Application No. PCT/US2006/012254, filed Mar. 31, 2006, and International Application No. PCT/US2008/065550, filed Jun. 2, 2008, all of which are incorporated herein in their entirety.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

What is claimed is:

1. A system for non-contact vibration monitoring, comprising:
at least two radio frequency (RF) transmitters for transmitting RF signals of a corresponding at least two wavelengths toward a target;
at least two RF receivers for detecting reflected RF signals that are reflected by the target; and
a processor for extraction of vibrational information regarding the target from the detected reflected electromagnetic signals.

2. The system according to claim 1, wherein the at least two RF transmitters are capable of simultaneously transmitting RF signals and the at least two RF detectors are capable of simultaneously detecting reflected RF signals.

3. The system according to claim 1, wherein the at least two RF transmitters are capable of sequentially transmitting RF signals and the at least two RF detectors are capable of sequentially detecting reflected RF signals.

4. The system according to claim 3, wherein the at least two RF transmitters are a single RF transmitter capable of sequentially transmitting at least two wavelengths.

5. The system according to claim 4, wherein the at least two RF receivers are a single RF receiver capable of sequentially detecting reflected RF signals at the at least two wavelengths.

6. The system according to claim 5, wherein the single RF receiver is capable of scanning over a range of RF wavelengths.

7. The system according to claim 1, wherein the vibration information includes the frequency of a vibration of the target.

8. The system according to claim 1, wherein the vibration information includes the magnitude of a vibration of the target.

9. The system according to claim 7, wherein the vibration information includes the magnitude of a vibration of the target.

10. The system according to claim 8, wherein the magnitude of the vibration of the target is in the range of λ₁/20 to λ₁/5 or λ₂/20 to λ₂/5, where λ₁ and λ₂ are two of the at least two wavelengths.

11. The system according to claim 1, wherein vibrations of the target cause a phase shift in the reflected RF signals.

12. The system according to claim 1, wherein the at least two RF transmitters and the at least two RF receivers comprise at least two RF transceivers.

13. The system according to claim 1, wherein the processor extracts vibrational information regarding the target by extracting harmonics of each of the least two wavelengths from the detected reflected signals.

14. The system according to claim 1, wherein the target is a human, wherein the at least two wavelengths are selected such that information regarding the target's cardiopulmonary activity and information regarding the target's respiratory activity are extracted.

15. The system according to claim 14, wherein the frequency of the target's cardiopulmonary activity and the frequency of the target's respiratory activity are extracted.

16. The system according to claim 14, wherein the magnitude of the target's cardiopulmonary activity and the magnitude of the target's respiratory activity are extracted.

17. The system according to claim 16, wherein the frequency of the target's cardiopulmonary activity and the frequency of the target's respiratory activity are extracted.

18. The system according to claim 1, wherein the at least two RF transmitters comprise at least two of the following:
an S-band transmitter,
a C-band transmitter,
an X-band transmitter,
a K-band transmitter, and
a Ka-band transmitter.

19. The system according to claim 12, wherein the at least two RF transceivers comprise at least two of the following:
an S-band transceiver,
a C-band transceiver,
an X-band transceiver,
a K-band transceiver, and
a Ka-band transceiver.

20. The system according to claim 1, wherein the at least two RF transmitters comprise:
an S-band transmitter,
a C-band transmitter,
an X-band transmitter,
a K-band transmitter, and
a Ka-band transmitter.

21. The system according to claim 1, wherein the extracted vibrational information allows monitoring of a corresponding at least two vibrations of the target having a corresponding at least two amplitudes.

22. The system according to claim 1, wherein the processor extracts vibrational information via a wavelength division technique.

23. The system according to claim 1, wherein the system allows monitoring of a complex movement pattern of the target.

24. A method for non-contact vibration monitoring, comprising:
transmitting at least two RF signals of a corresponding at least two wavelengths toward a target;
detecting at least two reflected RF signals that are reflected by the target; and
processing the detected at least two reflected RF signals to extract vibrational information regarding the target.

25. The method according to claim 24, wherein the at least two RF signals are transmitted simultaneously and the at least two reflected RF signals are detected simultaneously.

26. The method according to claim 24, wherein the at least two RF signals are transmitted sequentially and the at least two reflected RF signals are detected sequentially.

27. The method according to claim 26, wherein the at least two RF signals are transmitted by a single RF transmitter capable of sequentially transmitting at least two wavelengths.

28. The method according to claim 27, wherein the at least two reflected RF signals are detected by a single RF receiver capable of sequentially detecting reflected RF signals at the at least two wavelengths.

29. The method according to claim 28, wherein the single RF receiver is capable of scanning over a range of RF wavelengths, further comprising transmitting an RF signal over a range of wavelengths.

30. The method according to claim 24, wherein the vibration information includes the frequency of a vibration of the target.

31. The method according to claim 24, wherein the vibration information includes the magnitude of a vibration of the target.

32. The method according to claim 30, wherein the vibration information includes the magnitude of a vibration of the target.

33. The method according to claim 31, wherein the magnitude of the vibration of the target is in the range of λ₁/20 to λ₁/5 or λ₂/20 to λ₂/5, where λ₁ and λ₂ are two of the at least two wavelengths.

34. The method according to claim 24, wherein vibrations of the target cause a phase shift in the reflected RF signals.

35. The method according to claim 24, wherein transmitting the at least two RF signals and detecting the at least two RF reflected signals is accomplished via a corresponding at least two RF transceivers.

36. The method according to claim 24, wherein processing the detected at least two reflected RF signals extracts vibrational information regarding the target by extracting harmonics of each of the least two wavelengths from the detected reflected signals.

37. The method according to claim 24, wherein the target is a human, wherein the at least two wavelengths are selected such that information regarding the target's cardiopulmonary activity and information regarding the target's respiratory activity are extracted.

38. The method according to claim 37, wherein the frequency of the target's cardiopulmonary activity and the frequency of the target's respiratory activity are extracted.

39. The method according to claim 37, wherein the magnitude of the target's cardiopulmonary activity and the magnitude of the target's respiratory activity are extracted.

40. The method according to claim 39, wherein the frequency of the target's cardiopulmonary activity and the frequency of the target's respiratory activity are extracted.

41. The method according to claim 24, wherein the at least two RF signals comprise at least two of the following:
an S-band signal,
a C-band signal,
an X-band signal,
a K-band signal, and
a Ka-band signal.

42. The method according to claim 24, wherein the at least two RF signals comprise:
an S-band signal,
a C-band signal,
an X-band signal,
a K-band signal, and
a Ka-band signal.

43. The method according to claim 24, wherein the extracted vibrational information allows monitoring of a corresponding at least two vibrations of the target.

44. The method according to claim 24, wherein processing the detected at least two reflected RF signals extracts vibrational information via a wavelength division technique.

45. The method according to claim 24, wherein the method allows monitoring of a complex movement pattern of the target.
